(19) United States
(12) Patent Application Publication
(10) Pub. No.: US 2018/0226838 A1
(43) Pub. Date: Aug. 9, 2018

(54) PHASED ARRAY ULTRASONIC TRANSMITTER FOR DETECTING AND POWERING A WIRELESS POWER RECEIVER

(71) Applicant: QUALCOMM Incorporated, San Diego, CA (US)

(72) Inventor: Arvind GOVINDARAJ, San Diego, CA (US)

(21) Appl. No.: 15/424,142

(22) Filed: Feb. 3, 2017

(51) Int. Cl. H02J 50/15; A61B 8/08; A61B 8/00; A61M 5/142; A61N 1/372; A61N 1/378; G01S 15/42

(52) U.S. Cl. CPC H02J 50/15; A61B 8/0841; A61B 8/4488; A61M 5/14276; A61M 2205/825; A61N 1/3787; G01S 15/42; A61M 2205/353; A61N 1/37217

(57) ABSTRACT

Certain aspects of the present disclosure relate to methods and apparatus for detecting and powering a wireless receiver. An exemplary method generally includes scanning an area using a first plurality of ultrasonic pressure waves emitted from ultrasonic transducers on an ultrasonic phased array of ultrasonic transducers, detecting a first device in the area and determining a location of the first device in the area based on the scanning, and delivering a second plurality of ultrasonic pressure waves to the location of the first device in the area for powering the first device using the ultrasonic phased array of ultrasonic transducers.

### TECHNICAL FIELD

The present disclosure relates generally to wireless power transfer, and in particular to a phased array ultrasonic transmitter for detecting and powering a wireless power receiver.

### BACKGROUND

An increasing number and variety of electronic devices are powered via rechargeable batteries. Such devices include mobile phones, portable music players, laptop computers, tablet computers, computer peripheral devices, communication devices (e.g., Bluetooth® devices), digital cameras, hearing aids, medical implants, and the like. While battery technology has improved, battery-powered electronic devices increasingly require and consume greater amounts of power. As such, these devices constantly require recharging. Rechargeable devices are often charged via wired connections that require cables or other similar connectors that are physically connected to a power supply. Cables and similar connectors may sometimes be inconvenient or cumbersome and have other drawbacks. Wireless power transfer systems, for example, may allow users to charge and/or power electronic devices without physical, electrical connections, thus reducing the number of components required for operation of the electronic devices and simplifying the use of the electronic device.

For example, some battery powered devices, such as medical implants (e.g., pacemakers, neuromodulation devices, insulin pumps, etc.) may be located/positioned in areas where replacing the battery is not always feasible (e.g., in a body, such as, a human body). For example, to change a battery for a medical implant, surgery may need to be performed, which is risky. Accordingly, it may be safer to charge such devices wirelessly.

Further, some electronic devices may not be battery powered, but it still may be beneficial to utilize wireless power transfer to power such devices. In particular, the use of wireless power may eliminate the need for cords/cables to be attached to the electronic devices, which may be inconvenient and aesthetically displeasing.

Different electronic devices may have different shapes, sizes, and power requirements. There is flexibility in having different sizes and shapes in the components that make up a wireless power transmitter and/or a wireless power receiver in terms of industrial design and support for a wide range of devices

### SUMMARY

Certain aspects of the present disclosure provide a method for detecting and powering a wireless power receiver. An exemplary method generally includes scanning an area using a first plurality of ultrasonic pressure waves emitted from ultrasonic transducers on an ultrasonic phased array of ultrasonic transducers, detecting a first device in the area and determining a location of the first device in the area based on the scanning, and delivering a second plurality of ultrasonic pressure waves to the location of the first device in the area for powering the first device using the ultrasonic phased array of ultrasonic transducers.

Certain aspects of the present disclosure provide an apparatus for controlling an ultrasonic phased array of ultrasonic transducers for powering a wireless power receiver. An exemplary apparatus generally includes an ultrasonic phased array of ultrasonic transducers configured to scan an area by emitting a first plurality of ultrasonic pressure waves into the area, at least one processor configured to detect a first device in the area and determine a location of the first device in the area based on the scanning, and wherein the ultrasonic phased array of ultrasonic transducers is further configured to deliver a second plurality of ultrasonic pressure waves to the location of the first device in the area for powering the first device.

Certain aspects of the present disclosure provide an apparatus for detecting and powering a wireless power receiver. The apparatus generally includes means for scanning an area using a first plurality of ultrasonic pressure waves, means for detecting a first device in the area and determining a location of the first device in the area based on the scanning, and means for delivering a second plurality of ultrasonic pressure waves to the location of the first device in the area for powering the first device.

Certain aspects of the present disclosure provide a non-transitory computer-readable medium for detecting and powering a wireless power receiver. The non-transitory computer-readable medium generally includes instructions for scanning an area using a first plurality of ultrasonic pressure waves emitted from ultrasonic transducers on an ultrasonic phased array of ultrasonic transducers, detecting a first device in the area and determining a location of the first device in the area based on the scanning, and delivering a second plurality of ultrasonic pressure waves to the location of the first device in the area for powering the first device using the ultrasonic phased array of ultrasonic transducers.

The following detailed description and accompanying drawings provide a better understanding of the nature and advantages of the present disclosure.

### BRIEF DESCRIPTION OF THE DRAWINGS

With respect to the discussion to follow and in particular to the drawings, it is stressed that the particulars shown represent examples for purposes of illustrative discussion, and are presented in the cause of providing a description of principles and conceptual aspects of the present disclosure. In this regard, no attempt is made to show implementation details beyond what is needed for a fundamental understanding of the present disclosure. The discussion to follow, in conjunction with the drawings, makes apparent to those of skill in the art how embodiments in accordance with the present disclosure may be practiced. In the accompanying drawings:

FIG. 1 is a functional block diagram of a wireless power transfer system, according to certain aspects of the present disclosure.

FIG. 2 illustrates an ultrasonic phased array, according to certain aspects of the present disclosure.

FIG. 3 illustrates an example scenario in which an ultrasonic phased array structure may be used to determine locations of and transmit power to medical implants within a body, according to certain aspects of the present disclosure.

FIG. 4 illustrates a functional block diagram illustrating an implant detection and wireless power transfer system, according to certain aspects of the present disclosure.

FIG. 4A is a functional block diagram illustrating aspects of an implant detection module, according to certain aspects of the present disclosure.

FIG. 5A is a graph illustrating a passive implant detection scheme, according to certain aspects of the present disclosure.

FIG. 5B is a graph illustrating an active implant detection scheme, according to certain aspects of the present disclosure

FIG. 6 is a flow chart illustrating an implant detection process, according to aspects of the present disclosure.

FIG. 7 illustrates a functional block diagram of an implant capable of being powered by ultrasonic energy transfer, according to aspects of the present disclosure.

FIG. 8 illustrates example operations for detecting and powering medical implants, according to aspects of the present disclosure.

Drawing elements that are common among the following figures may be identified using the same reference numerals.

### DETAILED DESCRIPTION

As noted above, certain battery powered devices, such as medical implants (e.g., pacemakers, neuromodulation devices, insulin pumps, etc.) may be located/positioned in areas where replacing the battery is not always feasible (e.g., in a body, such as, a human body). For example, to change a battery for a medical implant, surgery may need to be performed, which is risky. Accordingly, it may be safer to charge such devices wirelessly. For example, in some cases, ultrasonic (pressure waves) energy transfer may be used to determine a location of and wirelessly power a wireless power receiver, such as a medical implant.

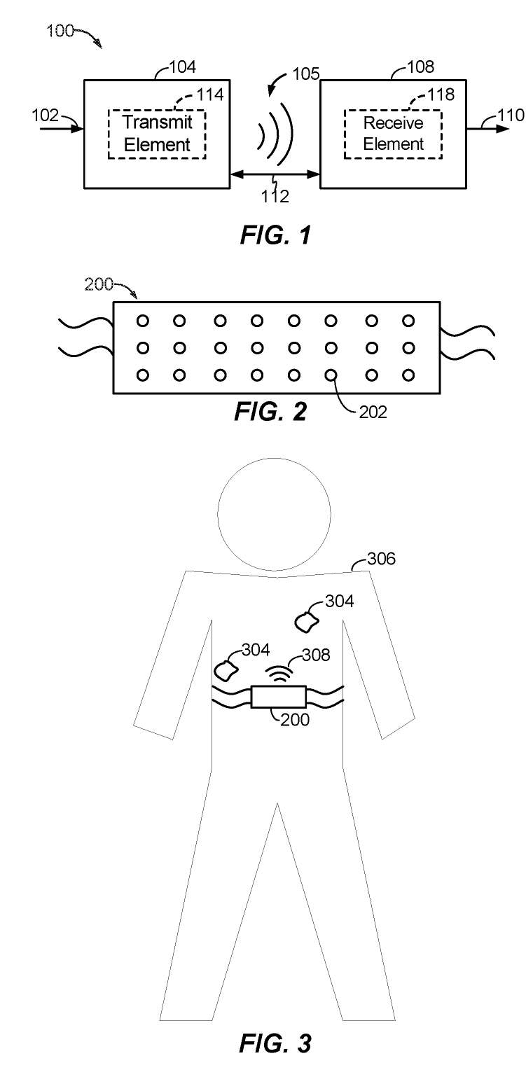

FIG. 1 is a functional block diagram of a wireless power transfer system 100, in accordance with certain aspects of the present disclosure. Input power 102 may be provided to a transmitter 104 (e.g., an ultrasonic phased array comprising a plurality of ultrasonic transducers) from a power source (e.g., power supply, battery, mains electric power, etc., not shown in this figure) to generate ultrasonic pressure waves 105 for performing energy transfer. In particular, the transmitter 104 converts electrical energy in the form of input power 102 to mechanical energy in the form of ultrasonic pressure waves 105 through the use of ultrasonic transducers. The ultrasonic transducers may include conductive diaphragms which oscillate/vibrate to produce ultrasonic pressure waves 105 when exposed to electrostatic fields generated by conductive plates of the ultrasonic transducers based on input power 102.

A receiver 108 (e.g., an ultrasonic transducer) may receive the ultrasonic pressure waves 105 and generate output power 110 for storing or consumption by a device (e.g., a medical implant, not shown in this figure) coupled to the output power 110. For example, the ultrasonic pressure waves 105 produced by transmitter 104 may be incident on an ultrasonic transducer of receiver 108. A conductive diaphragm of the ultrasonic transducer of receiver 108 may vibrate/oscillate due to the incident ultrasonic pressure waves 105 and generate an electric field through interaction with a conductive plate of the ultrasonic transducer. The transmitter 104 and the receiver 108 may be separated by a distance 112. The transmitter 104 may include an ultrasonic pressure wave transmitting element 114 (e.g., ultrasonic transducers) for transmitting ultrasonic pressure waves to the receiver 108. The receiver 108 may include an ultrasonic pressure wave receiving element 118 (e.g., ultrasonic transducer) for receiving the ultrasonic pressure waves and converting these waves into usable electric power.

FIG. 2 illustrates an example ultrasonic phased array structure 200 that may be used to determine a location of and wirelessly power medical implants, according to certain aspects of the present disclosure. In some cases, the ultrasonic phased array structure 200 may be an example of the ultrasonic pressure wave transmitting element 114 illustrated in FIG. 1.

As illustrated, the ultrasonic phased array structure 200 may include a plurality of ultrasonic transducers 202 (e.g., piezo elements, capacitive micro-machined ultrasonic transducers, etc.), which are configured to both transmit ultrasonic pressure waves (e.g., into a body, such as a human body) and also receive reflected ultrasonic pressure waves (e.g., from the organs, tissues, implants, etc., in the body). According to aspects, the ultrasonic phased array structure 200 may be configured to implement an implant location system, which can detect a precise location of an implant in the body, for example, as described in greater detail below. According to aspects, once the location of the implant has been determined, the ultrasonic phased array structure 200 may be configured to deliver pressure waves (e.g., maximum-amplitude pressure waves) via the ultrasonic transducers 202 at the determined location, for example, using superposition described in greater detail below. The pressure waves may be converted by the implant into energy used to power the implant.

According to certain aspects, the ultrasonic phased array structure 200 may be body mounted in a variety of ways, such as mounted on a belt around the waist. Additionally, while FIG. 2 illustrates one example structure of an ultrasonic phased array structure 200, it should be understood that other structures may exist that accomplish the same purpose, for example, determining a location of a wirelessly powered medical implant and wirelessly powering the medical implant.

Though in certain aspects, location detection and wireless power transfer is described herein with respect to medical implants in the body, similar techniques may also be applicable for detecting other wireless power receivers in other objects or locations and wirelessly powering such detected wireless power receivers.

FIG. 3 illustrates an example scenario in which the ultrasonic phased array structure 200 may be used to determine locations of and transmit power to medical implants within a body. As illustrated, the ultrasonic phased array structure 200 may be mounted on a body 306 using a belt around the waist of the body 306, as noted above.

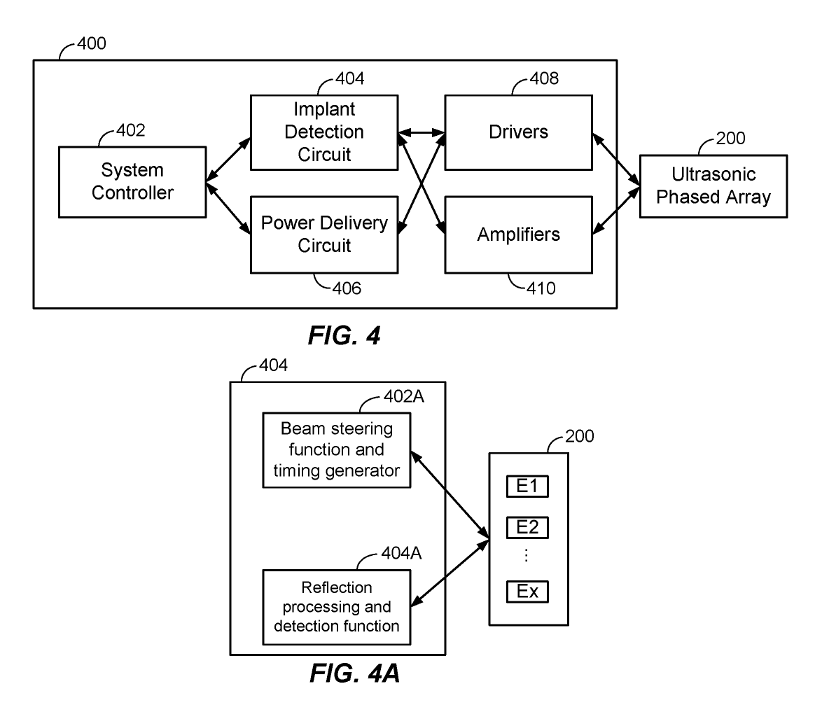

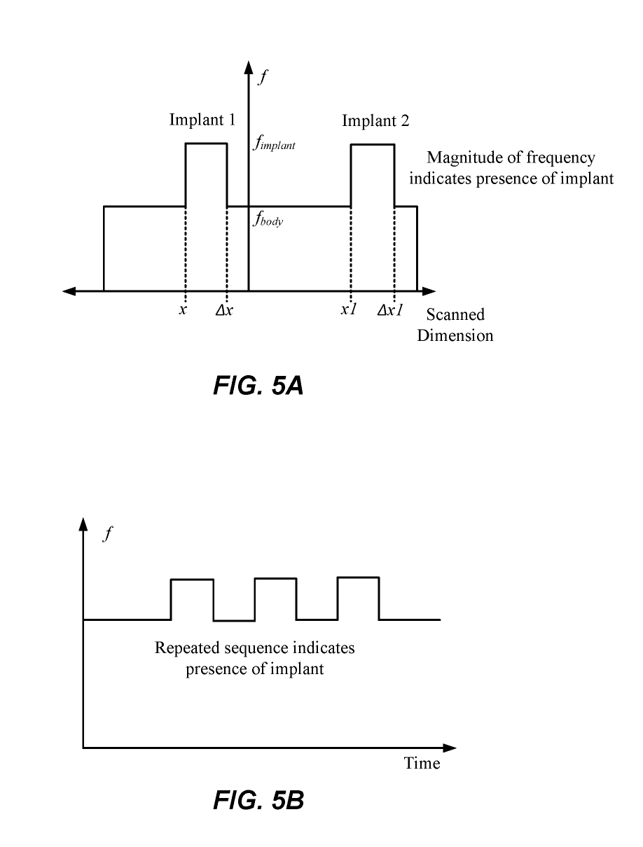

In the particular scenario illustrated in FIG. 3, when determining locations of medical implants (e.g., implants 304) within the body 306, the ultrasonic phased array structure 200 may transmit ultrasonic pressure waves 308 (e.g., via a plurality of ultrasonic transducers, not pictured) into the body 306. The ultrasonic phased array structure 200 may then listen for reflected waves within the body. According to certain aspects, based on the reflected waves, the locations of the implants 304 may be determined. Once the locations of the implants 304 within the body 306 are determined, the ultrasonic phased array structure 200 may again transmit ultrasonic pressure waves 308 into the body 306, beam-steering the ultrasonic pressure waves 308 towards the locations of the implants 304. The implants 304 may receive the ultrasonic pressure waves 308 and convert them into electric energy to power the implants 304. Aspects of the present disclosure related to determining the location of medical implants and delivering power (e.g., in the form of ultrasonic pressure waves) to these medical implants will be described in greater detail below with reference to FIGS. 4-5.

FIG. 4 is a functional block diagram illustrating an implant detection and wireless power transfer system 400, according to certain aspects of the present disclosure. As illustrated, the implant detection and wireless power transfer system 400 includes a system controller 402, an implant detection circuit 404 for detecting implants and their location inside a body (or similarly a device detection module for detecting other devices in any area), a power delivery circuit 406 for delivering power to determined implants, drivers 408 for coordinating communication between the implant detection circuit 404/power delivery circuit 406 and the ultrasonic phased array structure 200, and amplifiers 410 for amplifying received ultrasonic reflections from the ultrasonic phased array structure 200.

According to aspects, the system controller 402 may include one or more processors (not illustrated), such as a general purpose processor, a digital signal processor (DSP), an ASIC, a field programmable gate array (FPGA) or other programmable logic device (PLD), discrete gate or transistor logic, discrete hardware components, or any combination thereof designed to perform the functions described herein. In certain aspects, system controller 402 may be coupled to a memory configured to store instructions that when executed by system controller 402 perform the functions described herein. According to aspects, system controller 402 may be responsible for coordinating determination of the location of implants inside a body via the implant detection circuit 404 and power delivery to the implant via the power delivery circuit 406. For example, in some cases, the system controller 402 may receive an input instructing it to determine the location of an implant and deliver it power. The system controller 402 may communicate with the implant detection circuit 404, instructing the implant detection circuit 404 to determine a location of an implant inside a body, for example, as explained in greater detail below. Once the location of the implant inside the body is determined, the system controller 402 may then instruct the power delivery circuit 406 to deliver ultrasonic pressure waves (e.g., via the ultrasonic phased array structure 200) to the implant, which may then be converted by the implant into electric energy used to power the implant.

As noted, the implant detection circuit 404 is responsible for detecting implants in a body and their location within the body. According to aspects, the implant detection circuit 404 may detect implants within a body by listening for reflected sound waves generated based on an acoustic impedance mismatch between the human body and the materials that make up an implant. For example, to detect and determine a location of an implant, the ultrasonic phased array structure 200 may emit bursts (e.g., periodic bursts) of ultrasonic sound/pressure waves (synchronized between the plurality of ultrasonic transducers 202) and the implant detection circuit 404 may record the reflected sound waves (e.g., received via the ultrasonic phased array structure 200) generated in the body due to acoustic impedance mismatch. The orientation of the implant may also be determined using the intensity of reflected sound waves, as well as the angle of reflection. According to aspects, unlike traditional echo imaging, the impedance mismatch is expected to be greater than the mismatch between tissues, bones, and muscles, thereby facilitating easier and more accurate detection algorithms, for example, as explained below.

As noted, to detect and determine a location of an implant, the implant detection circuit 404 may send control signals to the drivers 408 to generate signals to drive the ultrasonic phased array structure 200 to scan a body (e.g., body 306) by emitting bursts (e.g., periodic bursts) of ultrasonic pressure waves, for example, using a process known as beam steering. According to certain aspects, scanning a body by beam steering ultrasonic pressure waves into the body involves using ultrasonic transducers (e.g., on the ultrasonic phased array structure 200) to individually generate chirps (e.g., lasting a few cycles) of ultrasound and transmit them into the body. In particular, the implant detection circuit 404 may send control signals to the drivers 408 that control the timing and strength of signals output by the drivers 408 to the ultrasonic transducers of the ultrasonic phased array structure 200 to separately control the ultrasonic transducers to generate chirps as described herein with reference to FIG. 4A.

FIG. 4A is a functional block diagram illustrating aspects of the implant detection circuit 404, according to certain aspects of the present disclosure. As illustrated, the implant detection circuit 404 may include a beam steering function and timing generator 402A and a reflection processing and detection function 404A. According to aspects, the beam steering function and timing generator 402A may interact with the ultrasonic phased array structure 200 (e.g., individual transducers or piezo elements E1-Ex on the ultrasonic phased array structure 200) to scan a body for implants, as described below. The reflection processing and detection function 404A may receive reflections from the body and determine if an implant is located within the body, as described in greater detail below.

According to aspects, the ultrasonic chirps transmitted from each individual transducer (e.g., piezo elements E1-Ex) may be time shifted, for example, by ΔTₓ, where ‘x’ corresponds to a particular transducer on the ultrasonic phased array structure 200. For example, the beam steering function and timing generator 402A may instruct a first ultrasonic transducer (e.g., piezo element E1) on the ultrasonic phased array structure 200 to generate and transmit a first ultrasonic chirp at time ΔT₁. The beam steering function and timing generator 402A may then instruct a second ultrasonic transducer (e.g., piezo element E2) to generate and transmit a second ultrasonic chirp at time ΔT₂, and so on. Each ΔTₓ may correspond to a time shift from when the detection process is started. According to aspects, the collective ultrasonic chirps generated by the plurality of transducers on the ultrasonic phased array structure 200 generate an ultrasonic wave-front whose direction may be controlled, thereby allowing the scanning of the body. For example, the direction of the ultrasonic wave-front produced by the ultrasonic phased array structure 200 may be controlled by selecting (e.g., by the beam steering function and timing generator 402A) a suitable ΔTₓ for each ultrasonic transducer. In some cases, the beam steering function and timing generator 402A may select the suitable ΔTₓ for each ultrasonic transducer according to Huygens's principle regarding reflections and refractions of waves. That is, using Hyugens's principle, beam steering function and timing generator 402A may determine a suitable ΔTₓ such that the ultrasonic wave-front may be steered, taking into account of individual chirps' reflections within the body. According to aspects, controlling the direction of the ultrasonic wave-front of allows the ultrasonic phased array structure 200 to direct ultrasonic chirps to different locations within the body thereby scanning an area of the body within range of the ultrasonic phased array structure 200. Additionally, an effective depth of field of the scanning beam (i.e., the ultrasonic wave-front) may be controlled by the beam steering function and timing generator 402A by varying ΔTₓ.

According to aspects, during scanning of the body, the ultrasonic wave-front transmitted/emitted by the ultrasonic phased array structure 200 may be reflected back due to a mismatch in acoustic impedances in the body (e.g., similar to impedance mismatch on electrical transmission lines). According to aspects, during transmission of the ultrasonic wave-front, incoming reflections may be received on ultrasonic transducers on the ultrasonic phased array structure 200. The received reflections may be amplified by amplifiers 410 and passed through delay elements (e.g., which can be implemented in hardware, software, or a combination of the two) and applied to a processing function (e.g., a summing function, also referred to as a detection function) in the reflection processing and detection function 404A of the implant detection circuit 404, which returns a numerical quantity (e.g., an output of a detection function) corresponding to the magnitude of a received reflection with respect to a location where the ultrasonic wave-front is transmitted/emitted.

Additionally, as noted above, an orientation of the implant within the body may be determined based on intensity of reflected sound waves, as well as the angle of reflection. For example, during transmission of the ultrasonic wave-front, incoming reflections may be received on ultrasonic transducers on the ultrasonic phased array structure 200. Based on the intensity of reflections as well as an angle of reflection, the implant detection circuit 404 may determine the orientation of an implant within a body. For example, when an implant is lined up with the ultrasonic phased array structure 200 the intensity of the reflections may be greater as compared to an implant that is improperly oriented. That is, when an implant is improperly oriented (e.g., in relation to the ultrasonic phased array structure 200), for example, beyond a certain threshold (e.g., degrees), the intensity of reflections may be (significantly) less than the intensity of reflections of a properly oriented implant.

According to aspects, a first detection function used to detect implants within a body may be referred to as passive implant detection implemented by the implant detection circuit 404 (e.g., in the reflection processing and detection function 404A). With reference to passive implant detection, an implant (e.g., implant 304) is expected to be constructed out of titanium, ceramic, or some other hard substance, which has a high acoustic impedance (˜27.32 normalized units, for example,

$$( \frac{g}{{cm}^{2} s} ) * {10}^{5} )$$

as compared to human tissue and bone (<˜10 normalized units). According to aspects, this results in a large mismatch in acoustic impedance between reflections of ultrasonic chirps from human tissue and bone as compared to reflections from implants. During scanning, this large mismatch in acoustic impedance creates an echo/reflection of the transmitted ultrasonic wave-front of larger magnitude which can be detected (e.g., by the implant detection circuit 404) by analyzing the output of the detection function. In other words, the higher the acoustic impedance of the material, the higher the magnitude of the reflected ultrasonic chirp. Therefore, a received reflection from an implant has a higher magnitude than received reflections from other parts of the body. Thus, positions in the body where a higher magnitude reflection is received (e.g., above a threshold, such as, baseline magnitude for the body) than reflections at other positions in the body may correspond to locations determined as having an implant. Thus, based on the magnitude of the reflections, an implant 304 in the body 306 may be detected and its location determined as discussed further below.

FIG. 5A is a magnitude response graph illustrating the passive implant detection scheme, described above. According to aspects, the curve illustrated in the magnitude response graph of FIG. 5A represents the output of the detection function, ‘ƒ’, of a particular reflection versus its location, ‘x’, within a larger scanned area. As illustrated, two implants may be detected in this particular scanned area based on the magnitudes of the reflections. For example, a first implant (e.g., Implant 1) may be detected (e.g., by the implant detection circuit 404) at a location between x and Δx as the magnitude of the reflections between these locations (i.e., x and Δx) is higher (e.g., satisfies a threshold magnitude, such as at a magnitude characteristic of a medical implant, ƒᵢₘₚₗₐₙₜ) as compared to the magnitude of reflections, for example, at locations less than x (e.g., that do not satisfy a threshold magnitude, such as at a magnitude characteristic of a human body, ƒ_{body}). Additionally, a second implant may be detected at a location between x1 and Δx1 as the magnitude of the reflections between these locations (i.e., x1 and Δx1) is higher (e.g., ƒᵢₘₚₗₐₙₜ) as compared to the magnitude of reflections, for example, at locations less than x (e.g., ƒ_{body}). Thus, as can be seen, based on the magnitude of reflections of ultrasonic chirps within a body, medical implants may be detected and their locations determined.

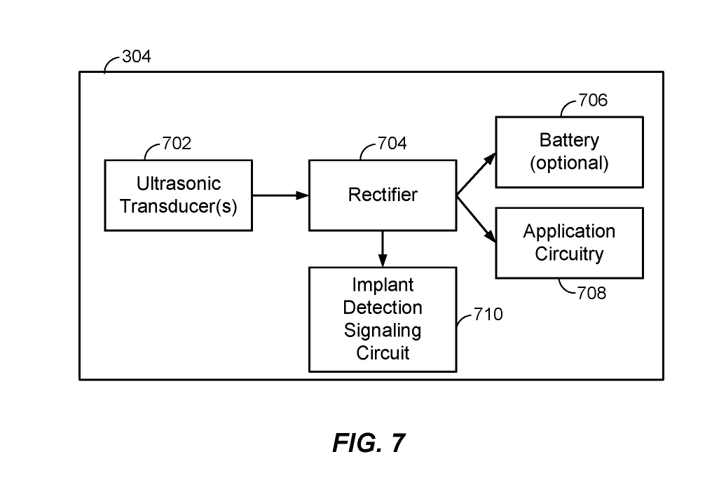

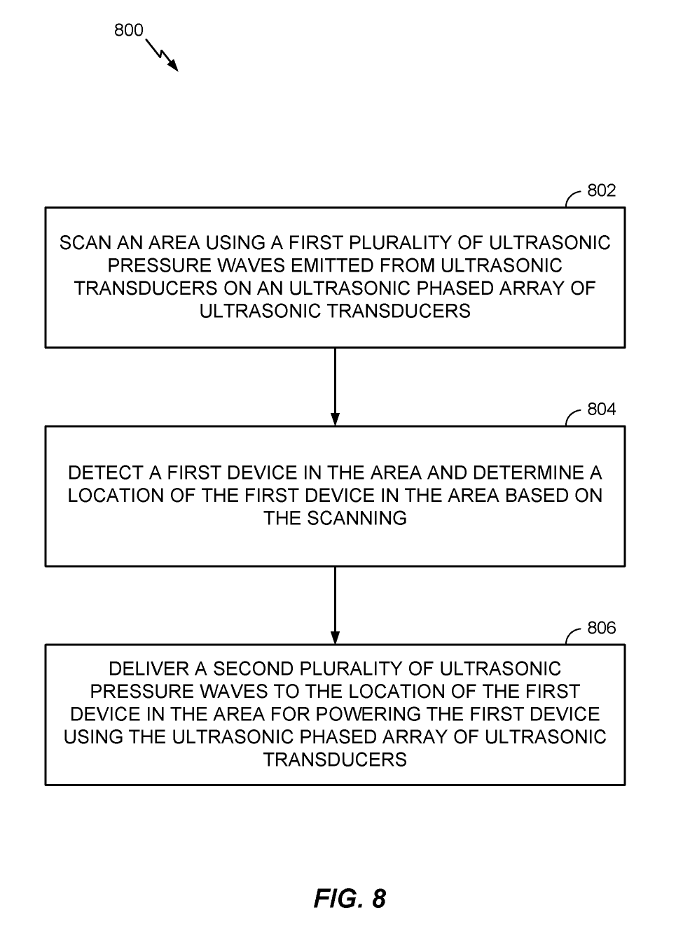

According to aspects, a second detection function may be referred to as active implant detection implemented by the implant detection circuit 404 (e.g., in the reflection processing and detection function 404A). According to certain aspects, an implant (e.g., implant 304) may include a signaling block (e.g., implant detection signaling circuit 710 as described below with respect to FIG. 7), which may include an active load that the signaling block controls to change the amount of power consumed by the active load. According to certain aspects, when the ultrasonic wave-front is incident on the implant, the signaling block in the implant may sink and/or modulate power by controlling the active load. Sinking power from the ultrasonic wave-front lowers the magnitude of the reflection/echo, while modulating power consumed from the ultrasonic wave-front modulates the magnitude of the reflection/echo, which may be detected by the implant detection circuit 404 as discussed. In order to detect the implant under active implant detection, the implant detection circuit 404 may analyze the magnitude of the reflections at a single spatial location over a period of time to see if the reflections (and their magnitudes) correspond to an expected pattern based on the sinking/modulation performed by the implant.

For example, the implant detection circuit 404 and implant may be pre-programmed or programmed to use a particular sinking/modulation pattern when the implant receives an ultrasonic wave-front. The implant detection circuit 404 may accordingly be configured to look for a pattern of reflections at a spatial location that corresponds to the particular sinking/modulation pattern. For example, in some aspects, the implant may be configured to modulate the power by periodically increasing and decreasing the power consumed by the active load according to a fixed pattern. Accordingly, the magnitude of the reflections received by the implant detection circuit 404 may also increase and decrease according to the fixed pattern (e.g., as seen in FIG. 5B). Therefore, if the implant detection circuit 404, when transmitting an ultrasonic wave-front to a particular spatial location, receives reflections according to the fixed pattern, the implant detection circuit 404 determines there is an implant at the spatial location.

In some aspects, the implant may sink power to decrease reflections below a threshold magnitude (e.g., below a threshold magnitude for other portions of the body). Accordingly, if the implant detection circuit 404 detects reflections with a magnitude that satisfies the threshold (e.g., is below the threshold), the implant detection circuit 404 determines there is an implant at the spatial location. In some aspects, the implant detection circuit 404 and implant may select from a plurality of fixed patterns or variable patterns, where each of the patterns correspond to different information. Accordingly, the implant can communicate different information to the implant detection circuit 404, such as identification information associated with the implant. In some aspects, the implant itself may actively transmit an implant detection signal (e.g., ultrasonic pressure waves via one or more ultrasonic transducers) modulated as described.

FIG. 5B is a graph illustrating the active implant detection scheme, described above. According to aspects, when scanning the body, the implant detection circuit 404 may see an output of the detection function, ‘ƒ’, similar to that illustrated in FIG. 5B. As illustrated, the output of the detection function contains a pattern (e.g., sequence of pulses) over a period of time, which may be a result of power sinking/modulation at the implant. The implant detection circuit 404 may identify this pattern in the output of the detection function and determine that an implant is detected. Additionally, in some cases, the pattern may comprise an implant detection signal that may be received by the implant detection circuit 404 and used to identify the implant.

According to aspects, while two different implant detection schemes (e.g., passive and active) are described separately, it should be understood that both of these implant detection schemes could be used in combination. Further, while aspects of the present disclosure generally describe detecting and identifying a (single) implant, it should be noted that the implant detection circuit 404 is capable of identifying multiple implants inside a body simultaneously, for example, using the implant detection schemes described above.

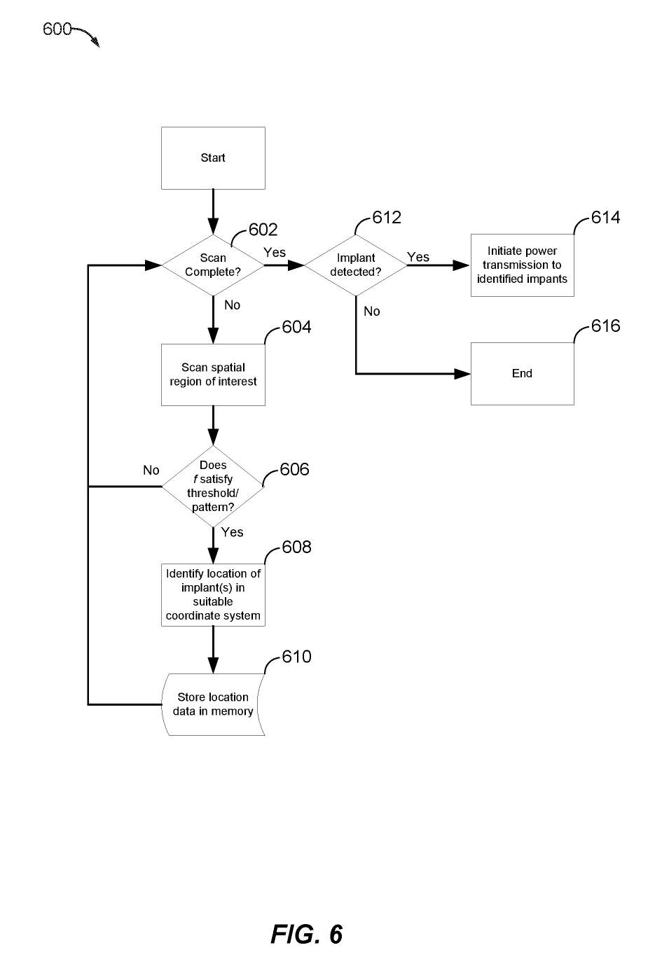

FIG. 6 is an example flow chart 600, illustrating an implant detection process, according to aspects of the present disclosure. According to aspects, the implant detection process illustrated in FIG. 6 may be performed, for example, by the implant detection circuit 404.

As illustrated, at 602, the implant detection circuit 404 determines whether a scan of a body (e.g., body 306) has already been completed. If a scan of the body has not been completed, the implant detection circuit 404 performs a scan of a spatial region (i.e., location) of interest on the body at 604. As noted, performing the scan of the body may involve beam-forming ultrasonic chirps emitted from the ultrasonic phased array structure 200, creating a ultrasonic wave-front, and controlling the direction of the ultrasonic wave-front to various spatial regions around the body, for example, as described above.

Once the scan of the body has been completed, the implant detection circuit 404 may analyze and output of a detection function for reflections received from the ultrasonic wave-front transmitted into the body at 606, for example, using one or more of the implant detection schemes described above. For example, in some cases, if the output of the detection function indicates a reflection magnitude that satisfies a threshold, the implant detection module may decide that an implant is detected within the body. Additionally, in some cases, the implant detection module may analyze the output of the detection function and may determine a pattern present in the output of the detection function. Based on the pattern the implant detection circuit 404 may determine an implant is detected within the body.

According to certain aspects, if the output of the detection function for received reflections does not satisfy the threshold or indicate a pattern of reflections, the implant detection circuit 404 may return to 602 and again determine whether a scan has been completed. However, if at 606 the implant detection circuit 404 determines that the output of the detection function for the received reflections does satisfy a threshold or indicates a pattern of reflections, the implant detection circuit 404 determines the location of the implant(s) in a suitable spatial coordinate system (e.g., cylindrical coordinates) at 608 as the location where the ultrasonic wave-front was directed. The implant detection circuit 404 may then store the determined coordinates of the implant(s) into memory at 610.

The process then returns to 602, where the implant detection module determines that a scan has been completed. Next, at 612, the implant detection circuit 404 determines whether an implant has been detected in the body. In some cases, this may involve the implant detection circuit 404 looking in memory to see if location coordinates of an implant are stored. According to aspects, if the implant detection circuit 404 determines that no implant was detected within the body, the process ends at 616. In some cases, another scan of the body may be performed at 604. If, however, the implant detection circuit 404 determines at 612 that an implant has been detected, the implant detection circuit 404 may instruct (e.g., via system controller 402) the power delivery circuit 406 to initiate power transmission to the identified implant(s) at 614.

Returning to FIG. 4, and as noted above, the implant detection and wireless power transfer system 400 includes a power delivery circuit 406 for delivering power to implants identified within the body. For example, if the implant detection circuit 404 determines the presence of an implant(s) within the body, the system controller 402 may instruct the power delivery circuit 406 to begin delivering ultrasonic pressure waves to the determined location(s) of the implant(s). For example, the power delivery circuit 406 may send control signals to the drivers 408 to generate signals to drive the ultrasonic phased array structure 200. For example the power delivery circuit 406 may send control signals to the drivers 408, instructing the ultrasonic phased array structure 200 of ultrasonic transducers, to beam-form ultrasonic chirps and direct a resultant ultrasonic wave-front to the determined location(s) of the implant(s). That is, the power delivery circuit 406 may direct the ultrasonic wave-front to the coordinates determined, for example, at 608. In particular, these control signals may control the timing and strength of signals output by the drivers 408 to the ultrasonic transducers of the ultrasonic phased array structure 200 to separately control the ultrasonic transducers to generate chirps as described herein.

According to certain aspects, if more than one implant is detected, the ultrasonic phased array structure 200 may be configured, in some cases, to time multiplex ultrasonic chirps directed to the implants. For example, at time T, the ultrasonic phased array structure 200 may transmit a first plurality of chirps that are beam-formed and directed to a first location of a first implant. At time T+1, the ultrasonic phased array structure 200 may transmit a second plurality of chirps that are beam-formed and directed to a second location of a second implant. The ultrasonic phased array structure 200 may then repeat this process, for example, by emitting the first plurality of chirps to the first location at T+2 and emitting the second plurality of chirps to the second location at T+3, and so on.

In other cases, the ultrasonic phased array structure 200 may be configured to spatially multiplex ultrasonic chirps directed to the implants. For example, ultrasonic phased array structure 200 may use a first plurality of ultrasonic transducers to deliver a first plurality of ultrasonic pressure waves to the location of the first implant and simultaneously use a second plurality of ultrasonic transducers to deliver a second plurality of ultrasonic pressure waves to the location of the second implant. According to aspects, the first plurality of ultrasonic pressure waves emitted from the first plurality of ultrasonic transducers may be beam-formed, as described above, and the beam-formed first plurality of ultrasonic pressure waves directed to the location of the first implant. For example, each of the first plurality of ultrasonic transducers may be spatially spaced apart and activated to emit a signal at the same time. However, the phase and/or intensity of the signal output by different ultrasonic transducers may be different based on the beam-forming performed. According to the superposition principal, the net response at a given location of the multiple signals from the first plurality of ultrasonic transducers is the sum of the responses of each signal at the given location. Accordingly, based on the different signals output by each the first plurality of ultrasonic transducers the, energy seen (e.g., received/delivered) at the first implant due to the first plurality of ultrasonic pressure waves may be more intense than at other locations in the body (e.g., locations other than where the first implant is), for example, based on the beam-forming/superposition principle. Additionally, the second plurality of ultrasonic pressure waves emitted from the second plurality of ultrasonic transducers (e.g., simultaneous with the first plurality of ultrasonic pressure waves) may be beam-formed and the beam-formed ultrasonic pressure waves directed to the location of the second implant. Likewise, energy seen (e.g., received/delivered) at the second implant due to the second plurality of ultrasonic pressure waves may be more intense than at other locations in the body (e.g., locations other than where the second implant is), for example, based on the beam-forming/superposition principle.

According to aspects, the implant(s) may receive ultrasonic pressure waves emitted (and beam-formed) by the ultrasonic phased array structure 200 and convert these ultrasonic pressure waves into electric energy to power the implant(s).

FIG. 7 is a functional block diagram of an implant 304 powered by ultrasonic energy transfer, according to aspects of the present disclosure. As illustrated, the implant 304 includes ultrasonic transducers 702, a rectifier 704, an optional battery 706, application circuitry 708, and an optional implant detection signaling circuit 710.

According to aspects, the ultrasonic transducers 702 may receive ultrasonic pressure waves from an ultrasonic phased array structure 200 of an implant detection and wireless power transfer system 400. According to aspects, these received ultrasonic pressure waves may be converted to AC electrical energy by the ultrasonic transducers 702. Further the rectifier 704 may convert the AC electrical energy to DC electrical energy for powering the implant 304. In some cases, the implant may include a battery 706, which may be used to store electric energy generated based on the received ultrasonic pressure waves. According to aspects, using the energy generated based on the received ultrasonic pressure waves, the implant 304 may run various applications programmed into the implant 304 using the application circuitry 708 (e.g., generating electric pluses at specific times to stimulate a heart, in the case of a pacemaker).

Additionally, in some cases, the implant 304 may include implant detection signaling circuit 710 (e.g., an active load). As noted above, the implant detection signaling circuit 710 may be configured to sink/modulate power from received ultrasonic pressure waves and/or transmit (e.g., via the ultrasonic transducers 702) an implant detection signal. According to aspects, the implant detection signal may be transmitted as one or more of the pulses in a sequence of ultrasonic pulses. As noted, the reflections/implant detection signal may be used by the implant detection and wireless power transfer system 400 to determine a presence and/or location of the implant 304.

FIG. 8 illustrates example operations 800 for detecting and powering a wireless power receiver, according to certain aspects of the present disclosure. According to certain aspects, operations 800 may be performed, for example, by a device detection and wireless power transfer system, such as implant detection and wireless power transfer system 400.

Operations 800 begin at 802 by scanning an area using a first plurality of ultrasonic pressure waves emitted from ultrasonic transducers on an ultrasonic phased array of ultrasonic transducers. At 804, the device detection and wireless power transfer system detects a first device in the area and determines a location of the first device in the area based on the scanning. For example, the first device may be a medical implant, and the area may be a body. According to aspects, detecting and determining the location of a first device may be performed according to one or more of the implant detection schemes described above, which may also be applied to detecting other types of devices in other areas.

At 806, the device detection and wireless power transfer system delivers a second plurality of ultrasonic pressure waves to the location of the first device in the area for powering the first device using the ultrasonic phased array of ultrasonic transducers. According to aspects, delivering ultrasonic pressure waves to the location of the first device may involve beam-forming individual ultrasonic pressure waves, and directing a resulting wave-front to the determined location of the first device, for example, as explained in greater detail above.

The various operations of methods described above may be performed by any suitable means capable of performing the corresponding functions. The means may include various hardware and/or software component(s) and/or module(s), including, but not limited to a circuit, an application-specific integrated circuit (ASIC), or processor. Generally, where there are operations illustrated in figures, those operations may have corresponding counterpart means-plus-function components with similar numbering.

For example, means for scanning, means for delivering, means for emitting, and means for receiving may comprise one or more ultrasonic transducers (e.g., ultrasonic transducers 202 on the ultrasonic phased array structure 200). Additionally, means for detecting, means for controlling, means for beam-forming, means for recording, means for determining, means for time-multiplexing, and/or means for using may comprise one or more processors (e.g., one or more processors in the system controller 402).

As used herein, the term “determining” encompasses a wide variety of actions. For example, “determining” may include calculating, computing, processing, deriving, investigating, looking up (e.g., looking up in a table, a database, or another data structure), ascertaining, and the like. Also, “determining” may include receiving (e.g., receiving information), accessing (e.g., accessing data in a memory), and the like. Also, “determining” may include resolving, selecting, choosing, establishing, and the like.

As used herein, a phrase referring to “at least one of” a list of items refers to any combination of those items, including single members. As an example, “at least one of: a, b, or c” is intended to cover: a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c).

The various illustrative logical blocks, modules and circuits described in connection with the present disclosure may be implemented or performed with a general purpose processor, a digital signal processor (DSP), an ASIC, a field programmable gate array (FPGA) or other programmable logic device (PLD), discrete gate or transistor logic, discrete hardware components, or any combination thereof designed to perform the functions described herein. A general-purpose processor may be a microprocessor, but in the alternative, the processor may be any commercially available processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

The functions described may be implemented in hardware, software, firmware, or any combination thereof. If implemented in hardware, an example hardware configuration may comprise a processing system in a wireless node. The processing system may be implemented with a bus architecture. The bus may include any number of interconnecting buses and bridges depending on the specific application of the processing system and the overall design constraints. The bus may link together various circuits including a processor, machine-readable media, and a bus interface. The bus interface may be used to connect a network adapter, among other things, to the processing system via the bus. The network adapter may be used to implement the signal processing functions of the physical (PHY) layer. In the case of a user terminal, a user interface (e.g., keypad, display, mouse, joystick, etc.) may also be connected to the bus. The bus may also link various other circuits such as timing sources, peripherals, voltage regulators, power management circuits, and the like, which are well known in the art, and therefore, will not be described any further.

The processing system may be configured as a general-purpose processing system with one or more microprocessors providing the processor functionality and external memory providing at least a portion of the machine-readable media, all linked together with other supporting circuitry through an external bus architecture. Alternatively, the processing system may be implemented with an ASIC with the processor, the bus interface, the user interface in the case of an access terminal), supporting circuitry, and at least a portion of the machine-readable media integrated into a single chip, or with one or more FPGAs, PLDs, controllers, state machines, gated logic, discrete hardware components, or any other suitable circuitry, or any combination of circuits that can perform the various functionality described throughout this disclosure. Those skilled in the art will recognize how best to implement the described functionality for the processing system depending on the particular application and the overall design constraints imposed on the overall system.

It is to be understood that the claims are not limited to the precise configuration and components illustrated above. Various modifications, changes and variations may be made in the arrangement, operation and details of the methods and apparatus described above without departing from the scope of the claims.

What is claimed is:

1. A method of powering a device, comprising:
scanning an area using a first plurality of ultrasonic pressure waves emitted from ultrasonic transducers on an ultrasonic phased array of ultrasonic transducers;
detecting a first device in the area and determining a location of the first device in the area based on the scanning; and
delivering a second plurality of ultrasonic pressure waves to the location of the first device in the area for powering the first device using the ultrasonic phased array of ultrasonic transducers.

2. The method of claim 1, wherein scanning comprises beam-forming the first plurality of ultrasonic pressure waves and directing the beam-formed first plurality of ultrasonic pressure waves to different locations in the area.

3. The method of claim 1, further comprising controlling a depth-of-field of the first plurality of ultrasonic pressure waves by varying a time at which individual ultrasonic pressure waves of the first plurality of ultrasonic pressure waves are emitted from the ultrasonic transducers.

4. The method of claim 1, wherein delivering the second plurality of ultrasonic pressure waves comprises selectively using ultrasonic transducers on the an ultrasonic phased array of ultrasonic transducers to deliver the second plurality of ultrasonic pressure waves to the location of the first device.

5. The method of claim 1, wherein delivering the second plurality of ultrasonic pressure waves comprises beam-forming the second plurality of ultrasonic pressure waves and directing the beam-formed second plurality of ultrasonic pressure waves to the location of the first device.

6. The method of claim 5, wherein beam-forming the second plurality of ultrasonic waves comprises time shifting at least a first ultrasonic pressure wave of the second plurality of ultrasonic pressure waves with respect to a at least a second ultrasonic pressure wave of the second plurality of ultrasonic pressure waves.

7. The method of claim 1, wherein scanning the area comprises:
emitting a burst of ultrasonic pressure waves into the area; and
recording reflections of the ultrasonic pressure waves in the area.

8. The method of claim 7, wherein detecting the first device and determining the location of the first device is based on acoustic impedance mismatch between reflections corresponding to the first device and reflections corresponding to other areas.

9. The method of claim 7, further comprising:
determining a pattern of the reflections over a period of time; and
wherein detecting the first device and determining the location of the first device is based on the pattern of the reflections.

10. The method of claim 7, further comprising determining an orientation of the first device based on an intensity of the reflections.

11. The method of claim 1, wherein the area comprises a body.

12. The method of claim 11, wherein the ultrasonic phased array of ultrasonic transducers comprises an apparatus worn on the body.

13. The method of claim 1, wherein the ultrasonic transducers comprise a piezo element or a capacitive micro-machined ultrasonic transducer (CMUT).

14. The method of claim 1, further comprising detecting additional devices in the area and determining locations in the area of the additional devices.

15. The method of claim 14, further comprising delivering at least a third plurality of ultrasonic pressure waves to the locations of the additional devices in the area for powering the additional devices, using the ultrasonic phased array of ultrasonic transducers.

16. The method of claim 15, wherein delivering the second plurality of ultrasonic pressure waves and the third plurality of ultrasonic pressure waves comprises at least one of:
time multiplexing individual ultrasonic pressure waves from the second plurality of ultrasonic pressure waves and the third plurality of ultrasonic pressure waves across the ultrasonic transducers of the ultrasonic phased array of ultrasonic transducers; or
using a first plurality of ultrasonic transducers of the ultrasonic phased array of ultrasonic transducers to deliver the second plurality of ultrasonic pressure waves to the first device and using a second plurality of ultrasonic transducers of the ultrasonic phased array of ultrasonic transducers to deliver the third plurality of ultrasonic pressure waves to the additional devices.

17. The method of claim 1, further comprising:
receiving a power-sinked reflection of the first plurality of ultrasonic pressure waves from the first device; and
wherein detecting the first device in the area and determining the location of the first device in the area is based on the power-sinked reflection.

18. The method of claim 1, wherein the first device comprises a medical implant, and wherein the area comprises a body.

19. An apparatus for controlling an ultrasonic phased array of ultrasonic transducers for powering a device, comprising:
an ultrasonic phased array of ultrasonic transducers configured to scan an area by emitting a first plurality of ultrasonic pressure waves into the area;
at least one processor configured to detect a first device in the area and determine a location of the first device in the area based on the scanning; and
wherein the ultrasonic phased array of ultrasonic transducers is further configured to deliver a second plurality of ultrasonic pressure waves to the location of the first device in the area for powering the first device.

20. The apparatus of claim 19, wherein the ultrasonic phased array of ultrasonic transducers is further configured to control a depth-of-field of the first plurality of ultrasonic pressure waves by varying a time at which individual ultrasonic pressure waves of the first plurality of ultrasonic pressure waves are emitted from the ultrasonic transducers.

21. The apparatus of claim 19, wherein the ultrasonic phased array of ultrasonic transducers is further configured to deliver the second plurality of ultrasonic pressure waves by beam-forming the second plurality of ultrasonic pressure waves and direct the beam-formed second plurality of ultrasonic pressure waves to the location of the first device.

22. The apparatus of claim 21, wherein beam-forming the second plurality of ultrasonic waves comprises time shifting at least a first ultrasonic pressure wave of the second plurality of ultrasonic pressure waves with respect to a at least a second ultrasonic pressure wave of the second plurality of ultrasonic pressure waves.

23. The apparatus of claim 19, wherein:
the ultrasonic phased array of ultrasonic transducers is configured to scan the area by emitting a burst of ultrasonic pressure waves into the area; and
the at least one processor is further configured to:
record reflections of the ultrasonic pressure waves in the area; and
detect the first device and determine the location of the first device based on acoustic impedance mismatch between reflections corresponding to the first device and reflections corresponding to other areas.

24. The apparatus of claim 23, wherein the at least one processor is further configured to:
determine a pattern of the reflections over a period of time; and
detect the first device and determine the location of the first device based on the pattern of the reflections.

25. The apparatus of claim 19, wherein:
the at least one processor is further configured to detect additional devices in the area and determine locations in the area of the additional devices; and
the ultrasonic phased array of ultrasonic transducers is further configured to deliver at least a third plurality of ultrasonic pressure waves to the locations of the additional devices in the area for powering the additional devices.

26. The apparatus of claim 25, wherein the ultrasonic phased array of ultrasonic transducers is further configured to deliver the second plurality of ultrasonic pressure waves and the third plurality of ultrasonic pressure waves by at least one of:
time multiplexing individual ultrasonic pressure waves from the second plurality of ultrasonic pressure waves and the third plurality of ultrasonic pressure waves across the ultrasonic transducers of the ultrasonic phased array of ultrasonic transducers; or
controlling a first plurality of ultrasonic transducers of the ultrasonic phased array of ultrasonic transducers to deliver the second plurality of ultrasonic pressure waves to the first device and controlling a second plurality of ultrasonic transducers of the ultrasonic phased array of ultrasonic transducers to deliver the third plurality of ultrasonic pressure waves to the additional devices.

27. The apparatus of claim 19, wherein:
the ultrasonic phased array of ultrasonic transducers is further configured to receive a power-sinked reflection of the first plurality of ultrasonic pressure waves from the first device; and
the at least one processor is further configured to detect the first device in the area and determine the location of the first device in the area is based on the power-sinked reflection.

28. The apparatus of claim 19, wherein the first device comprises a medical implant, and wherein the area comprises a body.

29. An apparatus for powering a device, comprising:
means for scanning an area using a first plurality of ultrasonic pressure waves;
means for detecting a first device in the area and determining a location of the first device in the area based on the scanning; and
means for delivering a second plurality of ultrasonic pressure waves to the location of the first device in the area for powering the first device.

30. A non-transitory computer-readable medium, comprising code that when executed by at least one processor causes the at least one processor to:
scan an area using a first plurality of ultrasonic pressure waves emitted from ultrasonic transducers on an ultrasonic phased array of ultrasonic transducers;
detect a first device in the area and determining a location of the first device in the area based on the scanning; and
deliver a second plurality of ultrasonic pressure waves to the location of the first device in the area for powering the first device using the ultrasonic phased array of ultrasonic transducers.
